(19) United States
(12) Patent Application Publication
(10) Pub. No.: US 2004/0111757 A1
(43) Pub. Date: Jun. 10, 2004

(54) Multiplex system for production and screening of monoclonal antibodies

(76) Inventors: Dongxiao Zhang, Moraga, CA (US); Robert Pytela, San Francisco, CA (US); Weimin Zhu, San Francisco, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/313,881

(22) Filed: Dec. 5, 2002

(51) Int. Cl. C12P 21/04

(52) U.S. Cl. 800/6; 435/70.21

(57) ABSTRACT

The invention provides methods for producing a plurality of monoclonal antibodies, which plurality contains an antibody for each member of a plurality of different antigens. In general, the methods involve administering a plurality of antigens or a cellular immunogen made up of a cell population expressing a plurality of antigens to a single animal to induce an immune response against the plurality of antigens, and producing monoclonal antibodies from antibody-producing cells of the animal. In general, the antigens expressed by the cells of the cellular immunogen are not native to the cells, and the cells are derived from the same species as the host. The invention further provides screening methods for identifying monoclonal antibodies of interest, and kits for carrying out the subject methods. The subjects systems, methods and kits find use in a variety of different industrial, medical and research applications.

### FIELD OF THE INVENTION

The field of this invention is monoclonal antibodies. The invention relates to methods of making monoclonal antibodies, particularly methods of making several monoclonal antibodies each reactive against a different antigen using a single host animal.

### BACKGROUND OF THE INVENTION

Monoclonal antibodies (Mabs) have been available for over 25 years and have revolutionalized biomedical research, especially in the areas of disease diagnosis and the treatment of infection and diseases.

The conventional method for the production of monoclonal antibodies involves hybridomas (Kohler & Milstein, Nature 256:495-7, 1975). In this method, splenic or lymphocyte cells from a mammal which has been injected with antigen are fused with a tumor cell line, thus producing hybrid cells. These hybrid cells, or “hybridomas”, are both immortal and capable of producing the genetically coded antibody of a B cell. To select a hybridoma producing a single antibody, the hybridomas made by cell fusion are segregated by selection, dilution, and regrowth until a single genetically pure antibody-expressing cell line is selected. Because hybridomas produce homogeneous antibodies against a desired antigen, they are called “monoclonal” antibodies. Hybridoma technology has primarily been focused on the fusion of murine lines, but also human-human hybridomas, human-murine hybridomas, rabbit-rabbit hybridomas and other xenogenic hybrid combinations have been made.

The arrival of the “genome era” of biology has brought new challenges to the scientific community, in that hundreds of thousands of encoded proteins have been identified, but their functions remain unknown. An important tool in understanding and manipulating the function of a protein is a monoclonal antibody that specifically binds to the protein. Since the scientific community is focusing significant effort on understanding the functions of these proteins, demands for monoclonal antibodies have reached record levels.

However, the production of monoclonal antibodies is usually costly and low-throughput.

Firstly, conventional methods commence with the preparation of a single purified antigen. Many antigens are difficult to express, purify and solubilize in amounts suitable for antibody production, and, as such, considerable time, expense, and effort is spent preparing enough antigen for a single immunization. Furthermore, most methods cannot be used to simultaneously express several antigens, so there are usually no economies of scale when several antigens are to be used.

Secondly, a single antigen is usually used to immunize a suitable host animal and therefore producing monoclonal antibodies for several antigens typically requires immunizing several hosts each with a single antigen. As such, when monoclonal antibodies are desired for several antigens, considerable time, expense, and effort is spent immunizing several animals.

Finally, making hybridoma cells and screening the cells for antibody production typically requires extensive selection for hybridoma cells that express antibodies to a single antigen. Because screening methods usually involve a single antigen, producing monoclonal antibodies for several antigens typically requires a very large amount of screening, which is also time consuming, expensive, and laborious.

As such, conventional monoclonal antibody production methods are laborious, expensive and slow throughput and cannot meet today's unprecedented demand for monoclonal antibodies. Accordingly, there is a great need for novel methods of monoclonal antibody production, particularly those methods that increasing throughput. The present invention addresses this, and other, needs.

Literature

The following papers may be of interest: McKenzie et al (Oncogene 4:543-8, 1989), Scuderi et al (Med. Oncol. Tumor Pharmacother 2:233-42, 1985), Roth et al (Surgery 96:264-72, 1984) and Drebin et al (Nature 312:545-8, 1984).

### SUMMARY OF THE INVENTION

The invention provides methods for producing a plurality of monoclonal antibodies, which plurality contains an antibody for each member of a plurality of different antigens. In general, the methods involve administering a plurality of antigens or a cellular immunogen made up of a cell population expressing a plurality of antigens to a single animal to induce an immune response against the plurality of antigens, and producing monoclonal antibodies from antibody-producing cells of the animal. In general, the antigens expressed by the cells of the cellular immunogen are not native to the cells, and the cells are derived from the same species as the host. The invention further provides screening methods for identifying monoclonal antibodies of interest, and kits for carrying out the subject methods. The subjects systems, methods and kits find use in a variety of different industrial, medical and research applications.

### BRIEF DESCRIPTION OF THE DRAWINGS

FIG. 1 is a photograph of immuno-peroxidase staining of a human oral carcinoma in situ with anti-integrin −β6 antibody. In pre-malignant lesions, integrin β6 staining is detected in in situ carcinoma using the rabbit monoclonal antibody B1, which detects αvβ6 with high affinity and specificity. A rabbit-specific secondary antibody was used and peroxidase staining indicates the presence of β6. We found that β6 displayed its characteristic basal localization at the tumor/stroma interface in tumors.

FIGS. 2a and 2b are photographs showing that antiserum from the rabbits immunized with 240E cells expressing Protein X gene specifically stains Protein X expressing cells. 240E cells were transiently transfected with expression plasmid containing CDNA of Protein X. After four rounds of immunizations, the antiserum (1:2000) was used to stain transfected 240E cells (FIG. 2a) and transfected 293 cells (FIG. 2b) with the same expression plasmid.

### DEFINITIONS

The terms “antibody” and “immunoglobulin” are used interchangeably herein. These terms are well understood by those in the field, and refer to a protein consisting of one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

An “antigen” is a substance that results in formation of antibodies that specifically bind to the substance when administered to an animal host.

The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized or murinized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the terms are Fab′, Fv, F(ab′)₂, and or other antibody fragments that retain specific binding to antigen.

Antibodies may exist in a variety of other forms including, for example, Fv, Fab, and (Fab′)₂, as well as bi-functional (i.e., bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988); Bird et al., Science, 242, 423-426 (1988); see Hood et al., “Immunology”, Benjamin, N.Y., 2nd ed. (1984), and Hunkapiller and Hood, Nature, 323, 15-16 (1986)).

An immunoglobulin light or heavy chain variable region consists of a “framework” region interrupted by three hypervariable regions, also called “complementarity determining regions” or CDRs. The extent of the framework region and CDRs have been precisely defined (see, “Sequences of Proteins of Immunological Interest,” E. Kabat et al., U.S. Department of Health and Human Services, (1983)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

Chimeric antibodies are antibodies whose light and heavy chain genes have been constructed, typically by genetic engineering, from antibody variable and constant region genes belonging to different species. For example, the variable segments of the genes from a rabbit monoclonal antibody may be joined to human constant segments, such as gamma 1 and gamma 3. An example of a therapeutic chimeric antibody is a hybrid protein composed of the variable or antigen-binding domain from a rabbit antibody and the constant or effector domain from a human antibody (e.g., the anti-Tac chimeric antibody made by the cells of A.T.C.C. deposit Accession No. CRL 9688), although other mammalian species may be used.

As used herein, unless otherwise indicated or clear from the context, antibody domains, regions and fragments are accorded standard definitions as are well known in the art. See, e.g., Abbas, A. K., et al., (1991) Cellular and Molecular Immunology, W. B. Saunders Company, Philadelphia, Pa.

As used herein, the term “humanized antibody” or “humanized immunoglobulin” refers to an antibody comprising one or more CDRs from an animal antibody, the antibody having been modified in such a way so as to be less immunogenic in a human than the parental animal antibody. An animal antibody can be humanized using a number of methodologies, including chimeric antibody production, CDR grafting (also called reshaping), and antibody resurfacing.

As used herein, the term “murinized antibody” or “murinized immunoglobulin” refers to an antibody comprising one or more CDRs from an animal antibody, the antibody having been modified in such a way so as to be less immunogenic in a mouse than the parental animal antibody. An animal antibody can be murinized using a number of methodologies, including chimeric antibody production, CDR grafting (also called reshaping), and antibody resurfacing.

It is understood that the humanized or murinized antibodies produced by the present method may have additional conservative amino acid substitutions which have substantially no effect on antigen binding or other antibody functions. By conservative substitutions is intended combinations such as gly, ala; val, ile, leu; asp, glu; asn, gln; ser, thr; lys, arg; and phe, tyr.

As used herein, the terms “determining,” “measuring,” and “assessing,” and “assaying” are used interchangeably and include both quantitative and qualitative determinations.

The terms “polypeptide” and “protein”, used interchangeably herein, refer to a polymeric form of amino acids of any length, i.e. greater than 2 amino acids, greater than about 5 amino acids, greater than about 10 amino acids, greater than about 20 amino acids, greater than about 50 amino acids, greater than about 100 amino acids, greater than about 200 amino acids, greater than about 500 amino acids, greater than about 1000 amino acids, greater than about 2000 amino acids, usually not greater than about 10,000 amino acids, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like. Also included by these terms are polypeptides that are post-translationally modified in a cell, e.g., glycosylated, cleaved, secreted, prenylated, carboxylated, phosphorylated, etc, and polypeptides with secondary or tertiary structure, and polypeptides that are covalently or non-covalently bound to other moieties, e.g., other polypeptides, atoms, cofactors, etc.

As used herein the term “isolated,” when used in the context of an isolated antibody, refers to an antibody of interest that is at least 60% free, at least 75% free, at least 90% free, at least 95% free, at least 98% free, and even at least 99% free from other components with which the antibody is associated with prior to purification.

A “coding sequence” or a sequence that “encodes” a selected polypeptide, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or procaryotic DNA, and synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence. “Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences that are immunologically identifiable with a polypeptide encoded by the sequence.

“Operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of a coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells, which can be accomplished by genomic integration of all or a portion of the vector, or transient or inheritable maintenance of the vector as an extrachromosomal element. Thus, the tern includes cloning, and expression vehicles, as well as integrating vectors.

An “expression cassette” comprises any nucleic acid construct capable of directing the expression of a gene/coding sequence of interest, which is operably linked to a promoter of the expression cassette. Such cassettes can be constructed into a “vector,” “vector construct,” “expression vector,” or “gene transfer vector,” in order to transfer the expression cassette into target cells. Thus, the term includes cloning and expression vehicles, as well as viral vectors.

Techniques for determining nucleic acid and amino acid “sequence identity” are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff 60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions that form stable duplexes between homologous regions, followed by digestion with single-stranded-specific nuclease(s), and size determination of the digested fragments. Two DNA, or two polypeptide sequences are “substantially homologous” to each other when the sequences exhibit at least about 80%-85%, preferably at least about 85%-90%, more preferably at least about 90%-95%, and most preferably at least about 95%-98% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., infra; DNA Cloning, supra; Nucleic Acid Hybridization, supra.

Two nucleic acid fragments are considered to “selectively hybridize” as described herein when they detectably pair with each other. The degree of sequence identity between two nucleic acid molecules affects the efficiency and strength of hybridization events between such molecules. A partially identical nucleic acid sequence will at least partially inhibit a completely identical sequence from hybridizing to a target molecule. Inhibition of hybridization of the completely identical sequence can be assessed using hybridization assays that are well known in the art (e.g., Southern blot, Northern blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. A nucleic acid molecule that is capable of hybridizing selectively to a target sequence under “moderately stringent” conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts, and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.). An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5× Denhardt's solution, 10% dextran sulfate, and 20 mg/ml denatured, sheared salmon sperm DNA, followed by washing the filters in 0.1×SSC at about 65° C. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed to identify nucleic acids of this particular embodiment of the invention.

A first polynucleotide is “derived from” a second polynucleotide if it has the same or substantially the same nucleotide sequence as a region of the second polynucleotide, its cDNA, complements thereof, or if it displays sequence identity as described above. A first polynucleotide may be derived from a second polynucleotide if the first polynucleotide is used as a template for, e.g., amplification of the second polynucleotide.

A first polypeptide is “derived from” a second polypeptide if it is (i) encoded by a first polynucleotide derived from a second polynucleotide, or (ii) displays sequence identity to the second polypeptides as described above. The term “unit dosage form” as used herein refers to physically discrete units suitable as unitary dosages for subjects (e.g., animals, usually humans), each unit containing a predetermined quantity of an agent, e.g., an antibody in an amount sufficient to produce the desired effect in association with a pharmaceutically acceptable diluent, carrier or vehicle. The specifications for the novel unit dosage forms of the present invention will depend on a variety of factors including, but not necessarily limited to, the particular agent employed and the effect to be achieved, and the pharmacodynamics associated with each compound in the host.

A polynucleotide is “derived from” a particular cell if the polynucleotide was obtained from the cell. A polynucleotide may also be “derived from” a particular cell if the polynucleotide was obtained from the progeny of the cell, as long as the polynucleotide was present in the original cell. As such, a single cell may be isolated and cultured, e.g., in vitro, to form a cell culture. A nucleotide isolated from the cell culture is “derived from” the single cell, as long as the nucleic acid was present in the isolated single cell.

A cell is “derived from” a host if the cell was obtained from the host. The progeny of a progenitor cell are derived from the same host as a progenitor cell. A cell may be “derived from” the same species as the host if the cell was isolated from an animal of the same species as the host animal. For example, NIH 3T3 cells are derived from mouse, 240E cells are derived from rabbit, and DT-40 cells are derived from chicken. The progeny of a progenitor cell are derived from the same species as the progenitor cell.

An antigen is “native” to a cell if the antigen is usually expressed by the cell. For example, rabbit 240E cells express rabbit polypeptide antigens. An antigen is “not-native” to a cell if the antigen is not usually expressed by the cell. For example, rabbit 240E cells do not usually express human polypeptide antigens, i.e., a polypeptide encoded by the human genome, and, as such, a human polypeptide is not native to a rabbit 240E cell.

The terms “treatment” “treating” and the like are used herein to refer to any treatment of any disease or condition in a mammal, e.g., particularly a human or a mouse, and includes: a) preventing a disease, condition, or symptom of a disease or condition from occurring in a subject which may be predisposed to the disease but has not yet been diagnosed as having it; b) inhibiting a disease, condition, or symptom of a disease or condition, e.g., arresting its development and/or delaying its onset or manifestation in the patient; and/or c) relieving a disease, condition, or symptom of a disease or condition, e.g., causing regression of the condition or disease and/or its symptoms.

The terms “subject,” “host,” “patient,” and “individual” are used interchangeably herein to refer to any mammalian subject for whom diagnosis or therapy is desired, particularly humans. Other subjects may include cattle, dogs, cats, guinea pigs, rabbits, rats, mice, horses, and so on.

Before the present subject invention is described further, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an antibody” includes a plurality of such antibodies and reference to “a variable domain” includes reference to one or more variable domains and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF PREFERRED EMBODIMENTS

The invention provides methods for producing a plurality of monoclonal antibodies, which plurality contains an antibody for each member of a plurality of different antigens. In general, the methods involve administering a cellular immunogen made up of a cell population expressing a plurality of antigens to a single animal to induce an immune response against the plurality of antigens, and producing monoclonal antibodies from antibody-producing cells of the animal. In general, the antigens expressed by the cells of the cellular immunogen are not native to the cells, and the cells are derived from the same species as the host. The invention further provides screening methods for identifying monoclonal antibodies of interest, and kits for carrying out the subject methods. The subjects systems, methods and kits find use in a variety of different industrial, medical and research applications.

In further describing the subject invention, the methods of the invention will be described first, followed by a review of the methods and representative applications in which the subject methods find use and kits/systems for practicing these applications. In one embodiment, the invention provides methods of making a cellular immunogen, which methods will be described first.

Immunogens

In general, the invention provides methods of making antibodies against a plurality of different antigens in a single animal. By production of antibodies against a plurality of different antigens is meant production of a population of antibodies, individual members of which cross-react with individual antigens of a plurality of different antigens. In other words, the population of antibodies contains individual antibodies that each cross react with a different antigen.

The methods usually involve administering a plurality of different isolated antigens to a single animal such that antibodies that bind at least a subset of the plurality of different antigens are made by the animal (i.e. greater than about 20%, greater than about 40%, greater than about 60%, greater than about 70%, greater than about 80% or even greater than about 90% or even greater than about 95%, up to 100% of the antigens). In most embodiments, the antigens are biopolymers such as different polypeptides, oligopeptides, proteins, protein fragments, nucleic acids, polysaccharides, carbohydrates, lipids or oils, other molecules such as small inorganic or organic molecules, or mixtures or modified variants thereof. The antigens may also be different cells, cell fractions or cell extracts or mixtures thereof. In most embodiments, single isolated antigens are usually prepared and mixed to form an antigen mixture that is administered to a suitable animal. In certain other embodiments, the plurality of antigens is made using a cellular immunogen, methods for making of which are described in detail below.

Cellular Immunogens

The invention further provides methods of making a cellular immunogen. In general, the methods involve producing a cell population that produces a plurality of different antigens.

In most embodiments, a plurality of different antigens is expressed in a population of host cells through introduction of a plurality of different nucleic acids encoding the antigens. In most embodiments, the nucleic acids are comprised within expression cassettes. Individual antigens, antigen-encoding nucleic acids, expression cassettes, host cells, modified host cells, etc., of a plurality of the same are further described below.

A plurality of different nucleic acids is usually transferred into a population of host cells such that the population of host cells produces the plurality of antigens, and individual cells may produce the antigens within the cell and secrete and/or surface target certain antigens. In certain embodiments, a single host cell may receive more than one antigen-encoding nucleic acid and may produce more than one antigen, whereas other cells may receive one antigen-encoding nucleic acid and produce one antigen. The population of cells, in general, is usually a mixture of single antigen and multiple antigen expressing cells, although populations in which single cells express single antigens and populations in which single cells express multiple antigens are also envisioned.

In one embodiment, the antigen encoding nucleic acids are nucleic acids identified as having a particular property, e.g., they may be differentially expressed in a disease such as a cancer, for example colon or breast cancer, or expressed in a certain tissue, or expressed at a certain time during normal or abnormal development, or encode polypeptides with certain activities, e.g. secreted polypeptides, membrane bound polypeptides, cell surface polypeptides, or have a certain other activity. Such nucleic acids may be identified using conventional gene expression (e.g., microarray), subtractive hybridization (see, e.g, J Cancer Res Clin Oncol. 1997;123:447-51 and Gene 2001 262:207-14), or conventional library screening technologies. Antigen encoding nucleic acids may also be unselected, meaning that they are not selected because of their expression pattern or activity of the antigen.

In certain embodiments, a plurality of expression cassettes containing different antigen-encoding nucleic acids is provided by a cDNA library, where the cDNA is cloned into a vector suitable for the expression the cDNA. Such vectors typically provide a promoter suitable for use in host cells operably linked to the cDNA, and as such, a plurality of such vectors will provide a plurality of expression cassettes for expression of different antigens in host cells. Exemplary vectors suitable for library construction include pCI from Promega (Madison, Wis.), Retro-X system from Clontech (Palo Alto, Calif.) and pCDNA3.1 from Invitrogen (Carlsbad, Calif.) and cDNA libraries in suitable vectors (e.g., retroviral expression libraries or plasmid expression libraries) can be purchased from Clontech (Palo Alto, Calif.) and Stratagene (La Jolla, Calif.) or from EdgeBiosystems (Gaithersburg, Md.). In certain embodiments, the vectors may contain secretion signal or cell surface targeting sequences, as described in further detail below.

By plurality is meant more than 1, for example more than 2, more than about 5, more than about 10, more than about 20, more than about 50, more than about 100, more than about 200, more than about 500, more than about 1000, more than about 2000, more than about 5000, more than about 10,000, more than about 20,000, more than about 50,000, more than about 100,000, usually no more than about 200,000. A “population” contains a plurality of items.

In many embodiments, the methods involve modifying host cells to make modified host cells that produce at least one antigen, e.g., at least one polypeptide, protein, fragment of a polypeptide or protein, post-translationally modified polypeptide etc. In certain embodiments, antigens are secreted from the modified host cells and/or targeted to the surface of the modified host cells such that antigenic epitopes of the polypeptide are presented on the outside of the cells. For example, a transmembrane receptor kinase may be targeted to the surface of an individual cell, and may present antigenic epitopes on the ligand binding domain on the outside of the cell.

In general, the host cells may be any cells suitable for expressing an antigen, however, in many embodiments, animal cells, for example mammalian, e.g., mouse, rabbit, hamster, human etc. or avian, e.g., chicken cells, are used since animal cells usually ensure correct post-transcriptional modification, correct protein targeting, and correct protein conformation. In certain embodiments, the host cell is a human cell (e.g., HeLa), a mouse cell (e.g., NIH 3T3), a chicken cell (e.g., DT-40) or a rabbit cell (e.g., 240E), etc.

In many embodiments, the antigens are not native to the host cells i.e., are not usually expressed in the host cells, and are usually derived from a different species than the host cells. For example, if a host cells are rabbit host cells, the antigens may be human or mouse polypeptides, and if the host cells are mouse host cell, the antigens may be a human or rabbit polypeptides.

In most embodiments, the modified host cells produce antigens in the modified host cells, which, in certain embodiments, may be post-transcriptionally and/or post-translationally modified in the modified host cells such that the polypeptides are cleaved, rearranged, covalently modified, e.g., glycosylated, phosphorylated, prenylated, acetylation, amidation, carbamylated, deamidation, famesylated, formylation, geranyl-geranylated, methylated, or myristoylated, etc.

In certain embodiments, the antigens are selected because of a certain property, for example their expression pattern in a cell in which the antigens are usually found. Selected antigens may be selected because they are induced or repressed by a certain condition, e.g., a disease such as a cancer, for example colon or breast cancer, in a certain tissue, or at a certain time during normal or abnormal development. In certain other embodiments, the polypeptide is unselected, which means that its function, expression pattern, or identity is unknown. The identity and/or function of the polypeptide may be known or unknown.

In most embodiments, a single antigen of the plurality of antigens is encoded by a nucleic acid encoding at least the primary structure of the antigen, and the antigen-encoding nucleic acid is introduced into a host cell to provide for production of the antigen. In certain embodiments the nucleic acid encoding the antigen is a CDNA, sometimes a full-length CDNA, which means that the cDNA encodes a full length polypeptide. The sequence of the nucleic acid may be determined, may be partially determined, or may be unknown, and the nucleic acid may be selected, e.g., based on its expression under certain conditions, or may be unselected. In most embodiments, the nucleic acid is derived from a different species from which the host cell is derived. As such, the modified host cell is usually a recombinant host cell, and the antigen is encoded by a nucleic acid. In certain embodiments, the antigen is a post-translationally modified antigen and may have associated secondary, tertiary or quaternary structures.

A single antigen of the plurality is usually expressed in the modified host cell using an expression cassette containing a nucleic acid sequence encoding the antigen. Expression cassettes, including suitable promoters (e.g., inducible promoters) terminators, enhancers, translation initiation signals, translational enhancers, are well known in the art, and are discussed in Ausubel, et al, (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.). Suitable promoters include SV40 elements, as described in Dijkema et al., EMBO J. (1985) 4:761; transcription regulatory elements derived from the LTR of the Rous sarcoma virus, as described in Gorman et al., Proc. Nat'l Acad. Sci USA (1982) 79:6777; transcription regulatory elements derived from the LTR of human cytomegalovirus (CMV), as described in Boshart et al., Cell (1985) 41:521; hsp70 promoters, (Levy-Holtzman,R. and 1. Schechter (Biochim. Biophys. Acta (1995) 1263: 96-98) Presnail, J. K. and M. A. Hoy, (Exp. Appl. Acarol. (1994) 18: 301-308)) and the like. The expression polynucleotide provides expression cassettes for expression of an antigen in a host cell. In most embodiments, each expression cassette is more than about 0.5 kb in length, more than about 1.0 kb in length, more than about 1.5 kb in length, more than about 2 kb in length, more than about 4 kb in length, more than about 5 kb in length, and is usually less than 10 kb in length.

The expression cassette may be linear, or encompassed in a circular vector, which may further comprise a selectable marker. Suitable vectors, e.g., viral and plasmid vectors, and selectable markers are well known in the art and discussed in Ausubel, et al, (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.). A variety of different genes have been employed as selectable markers, and the particular gene employed in the subject vectors as a selectable marker is chosen primarily as a matter of convenience. Known selectable marker genes include: the thimydine kinase gene, the dihydrofolate reductase gene, the xanthine-guanine phosporibosyl transferase gene, CAD, the adenosine deaminase gene, the asparagine synthetase gene, the antibiotic resistance genes, e.g., tetr, ampr, Cmr or cat, kanr or neor (aminoglycoside phosphotransferase genes), the hygromycin B phosphotransferase gene, and the like. Vectors may provide for integration into the host cell genome, or may be autonomous from the host cell genome.

In certain embodiments, the expression cassette further provides for secretion of the antigen from the modified host cell by producing an antigen operably linked to a secretion signal. In such embodiments, the antigen encoding nucleic acid may be operably linked to a secretion signal-encoding nucleic acid in the expression cassette, and transcription and subsequent translation of the nucleic acids provides for production of a fusion protein containing the antigen and the secretion signal. As such, the expression cassette can provide for secretion of an antigen from a host cell, which antigen is not usually expressed from the host cell. Suitable secretion signals and their encoding nucleic acid sequences include signal sequences from secreted proteins, for example, human IgGkappa, growth hormone or IL-2. Suitable secretory sequences include secretion signals from IL-2 (MYRMQLLSCIALSLALVTNS; Villinger et al., J. Immunol. 155:3946 (1995)), growth hormone (MATGSRTSLLLAFGLLCLPWLQEGSAFPT; Roskam et al., Nucleic Acids Res. 7:30 (1979)); preproinsulin (MALWMRLLPLLALLALWGPDPAAAFVN; Bell et al., Nature 284:26 (1980)); influenza HA protein (MKAKLLVLLYAFVAGDQI; Sekiwawa et al., PNAS 80:3563)), and the signal leader sequence from the secreted cytokine IL-4, which comprises the first 24 amino acids of IL-4 as follows: MGLTSQLLPPLFFLLACAGNFVHG. In many embodiments, the secretion signal is derived from the same species as the host cell.

In certain other embodiments, an expression cassette further provides for targeting of the antigen to the surface of the host cell by producing an antigen operably linked to a cell surface targeting polypeptide. In such embodiments, the antigen encoding nucleic acid may be operably linked to a cell surface targeting polypeptide-encoding nucleic acid in the expression cassette, and transcription and subsequent translation of the nucleic acids provides for production of a fusion protein containing the antigen and the cell surface targeting polypeptide. As such, the expression cassette can provide for targeting of an antigen to the surface of a host cell, which antigen is not usually presented on the surface of the host cell. Suitable cell surface targeting polypeptides and their encoding nucleic acid sequences may be those of, for example, transmembrane serine threonine or tyrosine kinase receptors. Suitable cell surface targeting signals and their encoding nucleic acid sequences include receptor transmembrane domains, such as the epidermal growth factor receptor (EGFR) transmembrane domain (Ullrich, A. et al. Nature 309: 418-425 (1984)). In many embodiments, the cell surface targeting sequence is derived from the same species as the host cell. Further examples of strategies for targeting of polypeptides in a cell or protein secretion may be found in U.S. Pat. No. 6,455,247.

Expression cassettes may be introduced into a host cell using a variety of methods, including viral infection, transfection, conjugation, protoplast fusion, electroporation, particle gun technology, calcium phosphate precipitation, direct microinjection, viral vector delivery, and the like. The choice of method is generally dependent on the type of cell being transformed and the circumstances under which the transformation is taking place (i.e., in vitro). A general discussion of these methods can be found in Ausubel, et al, Short Protocols in Molecular Biology, 3rd ed., Wiley & Sons, 1995.

In most embodiments, the antigen is a polypeptide, and the polypeptide is from about 9-about 15 amino acids in length, about 16 to about 40 amino acids in length, about 41 to about 60 amino acids in length, about 61 to about 100 amino acids in length, about 101 to about 200 amino acids in length, about 201 to about 300 amino acids in length, about 301 to about 400 amino acids in length, about 401 to about 500 amino acids in length, usually less than about 1000 amino acids in length.

In many embodiments, the cellular immunogen comprises more than about 1 more than about 10⁵, more than about 10⁶, more than about 10⁷, more than about 10⁸, and usually no more than about 10⁹ or 10¹⁰ cells.

Methods of Producing an Animal Host that Produces a Population of Antibodies Which Contains an Antibody for Members of a Plurality of Different Antigens

The invention further provides methods of producing an animal host that produces a population of antibodies which contains individual antibodies for individual antigens of a plurality of different antigens. In general, the methods involve immunizing an animal, for example a suitable mammal such as a mouse, rabbit or guinea pig, or a suitable avian, such as a chicken, with a antigen mixture or a cellular immunogen, as described above, such that the cellular immunogen produces a plurality of different antigens in the animal.

In many embodiments, the administered cellular immunogen provide for antigen secretion and/or targeting to the surface of the cell, allowing antibodies that specifically bind the antigens to be made by the animal. In most embodiments, the cells of the cellular immunogen are derived from the same species as the animal (e.g., if the animal is a rabbit, the cells may be 240E cells expressing a plurality of antigen), and are usually not immunogenic when administered to an animal unless it is a modified host cell, i.e., the host produces at least one antigen that that is not native to the cell. In certain embodiments, the cells (or the progeny of the cells) or progeny of the cells are capable of surviving in the animal, on average, for more than about 1 hr, more than about 2 hr, more than about 6 hr, more than about 12 hr, more than about 1 day, more than about 3 days, more than about 7 days, more than about 28 days and may even survive for the remaining life of the animal. In certain embodiments, the cells are immortalized cells, and cells may be administered as living cells. If the cells contain expression cassettes containing an inducible promoter, induction with an appropriate promoter activity inducer will induce expression of the plurality of antigens.

Methods of immunizing animal, including the adjuvants used, booster schedules, sites of injection, suitable animals, etc. are well understood in the art, e.g., Harlow et al. (Antibodies: A Laboratory Manual, First Edition (1988) Cold spring Harbor, N.Y.), and administration of living cells to animals has been described for several mammals, e.g., and birds, e.g., McKenzie et al (Oncogene 4:543-8, 1989), Scuderi et al (Med. Oncol. Tumor Pharmacother 2:233-42, 1985), Roth et al (Surgery 96:264-72, 1984) and Drebin et al (Nature 312:545-8, 1984).

In many embodiments more than about 10⁴, more than about 10⁵, more than about 10⁶, more than about 10⁷, more than about 10⁸, and usually no more than about 10⁹ or 10¹⁰ antigen cells are administered to provide for expression of a plurality of antigens.

After the animals have been immunized, the animals usually mount an immune response against the plurality of antigens, and the blood of such animals will normally contain polyclonal antisera that bind (e.g., by ELISA, western blot, etc.), depending on how the methods are performed, at least about 5%, at least about 10%, at least about 20%, at least about 30%, at least about 40%, at least about 50%, at least about 80%, usually not more than about 90% or 95% or 100% of the plurality of antigens. Binding affinities of the polyclonal antisera to the antigens may vary between antigen, but will generally be in the range of at least about 10⁶ M⁻¹ at least about 10⁷ M⁻¹, at least about 10⁸ M⁻¹, or at least about 10⁹ M⁻¹ to 10¹⁰ M⁻¹) for each antigen.

Methods of Making Monoclonal Antibodies

The invention provides methods of making a plurality of monoclonal antibodies that bind a different antigens from the immunized animals described above. In general, the methods involve immunizing an animal with an antigen mixture or a cellular immunogen, as described above, and, generating a population of antibody producing cells. In one embodiment, the population of cells is produced using hybridoma methods that well known to one of skill in the art (see, e.g., Harlow Antibodies: A Laboratory Manual, First Edition (1988) Cold Spring Harbor, N.Y.). In alterative embodiments, populations of cells expressing monoclonal antibodies may be made using phage display methods, or the methods of U.S. Utility patent application Ser. No. 10/266,387, filed Oct. 7, 2002.

In general, once the population of monoclonal antibody-producing cells is produced, the antibodies produced by the cells are screened using one or a combination of a variety of assays. In general, these assays are functional assays, and may be grouped as follows: assays that detect an antibody's binding affinity or specificity, and assays that detect the ability of an antibody to inhibit a process.

A monoclonal antibody identified as having a specific binding activity with an antigen, or an inhibitory activity is termed a monoclonal antibody of interest.

Subject monoclonal antibodies are not usually associated with viral sequences, and are usually have immunoglobulin heavy and light chains that have been “naturally paired” by the immune system of the host. As such, in most embodiments, the subject monoclonal antibodies essentially maintain the combination of heavy and light chains that are present in the antibody producing cell from which monoclonal antibody is derived.

In most embodiments, the population of monoclonal antibody cells is screened for binding to a single antigen (i.e., antigens that are not mixed with other antigens of the plurality of antigens) of the plurality of antigens. When monoclonal antibodies that bind to several antigens are desired, the population is usually screened several times, corresponding to the number of antigens, usually in parallel.

In certain embodiments, however, antigens may be pooled during the initial stages of the screening process, e.g., in groups of 2, about 4, about 8, about 12, about 16, about 48 or more, usually up to about 96. In these embodiments, a final “deconvolution” step may be added to the conventional screening methods in order to identify which antigen of the pool is bound by a particular antibody.

In an alternative embodiment, antigens are pooled in a 2-dimensional, 3-dimensional or 4-dimensional array in the initial stages of the screening process, and a “deconvolution” step may be used in order to identify which antigen of the pool is bound by a particular antigen.

In certain embodiment, monoclonal antibodies are isolated for 1% to about 10%, 1% to about 20%, 1% to about 30%, 1% to about 50%, 1% to about 70%, 1% to about 80%, 1% to about 90%, or 1 to about 95% or 100% of the plurality of antigens administered to an animal.

Binding Assays

In these assays, each monoclonal antibody produced by the population of antibody-producing cells is tested for its ability to bind specifically to a substrate. The term “specifically” in the context of antibody binding, refers to high avidity and/or high affinity binding of an antibody to a specific antigen i.e., a polypeptide, or epitope. In many embodiments, the specific antigen is an antigen (or a fragment or subfraction of an antigen) used to immunize the animal host from which the antibody-producing cells were isolated. Antibody specifically binding an antigen or fragment thereof is stronger than binding of the same antibody to other antigens. Antibodies which bind specifically to a polypeptide may be capable of binding other polypeptides at a weak, yet detectable, level (e.g., 10% or less of the binding shown to the polypeptide of interest). Such weak binding, or background binding, is readily discernible from the specific antibody binding to a subject polypeptide, e.g., by use of appropriate controls. In general, specific antibodies bind to an antigen with a binding affinity of 10⁻⁷ or more, e.g., 10⁻⁸ M or more (e.g., 10⁻⁹ M, 10⁻¹⁰, 10⁻¹¹, etc.). In general, an antibody with a binding affinity of 10⁻⁶ M or less is not useful in that it will not bind an antigen at a detectable level using conventional methodology currently used.

Typically, in performing a screening assay, antibody samples produced by a library of antibody producing host cells are deposited onto a solid support in a way that each antibody can be identified, e.g., with a plate number and position on the plate, or another identifier that will allow the identification of the host cell culture that produced the antibody.

The antibodies of the invention may be screened for immunospecific binding by any method known in the art. The immunoassays which can be used include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally involve lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4.degree. C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads).

Western blot analysis generally involves preparation of protein samples followed by electrophoresis of the protein samples in a polyacrylamide gel (e.g., 8%-20% SDS-PAGE depending on the molecular weight of the antigen), and transfer of the separated protein samples from the polyacrylamide gel to a membrane such as nitrocellulose, PVDF or nylon. Following transfer, the membrane is blocked in blocking solution (e.g., PBS with 3% BSA or non-fat milk), washed in washing buffer (e.g., PBS-Tween 20), and incubated with primary antibody (the antibody of interest) diluted in blocking buffer. After this incubation, the membrane is washed in washing buffer, incubated with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I), and after a further wash, the presence of the antigen may be detected. One of skill in the art would be knowledgeable as to the parameters that can be modified to increase the signal detected and to reduce the background noise.

ELISAs involve preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

Antibodies of the invention may be screened using immunocytochemisty methods on cells (e.g., mammalian cells, such as CHO cells) transfected with a vector enabling the expression of an antigen or with vector alone using techniques commonly known in the art. Antibodies that bind antigen transfected cells, but not vector-only transfected cells, are antigen specific.

In certain embodiments, however, the assay is an antigen capture assay, and an array or microarray of antibodies may be employed for this purpose. Methods for making and using microarrays of polypeptides are known in the art (see e.g., U.S. Pat. Nos. 6,372,483, 6,352,842, 6,346,416 and 6,242,266).

Inhibitor Assays

In certain embodiments, the assay measures the specific inhibition of an antibody to an interaction between a first compound and a second compound (e.g., two biopolymeric compounds) or specifically inhibits a reaction (e.g., an enzymatic reaction). In the interaction inhibition assay, one interaction substrate, usually a biopolymeric compound such as a protei,n e.g., a receptor, may be bound to a solid support in a reaction vessel. Antibody is added to the reaction vessel followed by a detectable binding partner for the substrate, usually a biopolymeric compound such as a protein, e.g., a radio-labeled ligand for the receptor. After washing the vessel, interaction inhibition may be measured by determining the amount of detectable binding partner present in the vessel. Interaction inhibition occurs when binding of the binding partner is reduced greater than about 20%, greater than about 50%, greater than about 70%, greater than about 80%, or greater than about 90% or 95% or more, as compared to a control assay that does not contain antibody.

In the reaction inhibition assay, an enzyme may be bound to a solid support in a reaction vessel. Antibody is usually added to the reaction vessel followed by a substrate for the enzyme. In many embodiments, the products of the reaction between the enzyme and the substrate are detectable, and, after a certain time, the reaction is usually stopped. After the reaction has been stopped, reaction inhibition may be measured by determining the level of detectable reaction product present in the vessel. Reaction inhibition occurs when the rate of the reaction is reduced greater than about 20%, greater than about 50%, greater than about 70%, greater than about 80%, or greater than about 90% or 95% or more, as compared to a control assay that does not contain antibody.

In Vivo Assays

In certain embodiments the monoclonal antibodies are tested in vivo. In general, the method involves administering a subject monoclonal antibody to an animal model for a disease or condition and determining the effect of the monoclonal antibody on the on the disease or condition of the model animal. In vivo assays of the invention include controls, where suitable controls include a sample in the absence of the monoclonal antibody. Generally a plurality of assay mixtures is run in parallel with different antibody concentrations to obtain a differential response to the various concentrations. Typically, one of these concentrations serves as a negative control, i.e., at zero concentration or below the level of detection.

A monoclonal antibody of interest is one that modulates, i.e., reduces or increases a symptom of the animal model disease or condition by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, when compared to a control in the absence of the antibody. In general, a monoclonal antibody of interest will cause a subject animal to be more similar to an equivalent animal that is not suffering from the disease or condition. Monoclonal antibodies that have therapeutic value that have been identified using the methods and compositions of the invention are termed “therapeutic” antibodies.

Methods of Identifying Nucleic Acids Encoding a Monoclonal Antibody of Interest

The invention further provides a method of identifying a nucleic acid encoding a monoclonal antibody of interest. In general, the method involves screening the plurality of monoclonal antibodies to identify an monoclonal antibody of interest; and identifying nucleic acids encoding the monoclonal antibody of interest.

Since the host cell expressing the antibody of interest contains the immunoglobulin heavy and light chain-encoding expression cassettes, the nucleic acids encoding the monoclonal antibody of interest may be identified if the host cell expressing the monoclonal antibody of interest is identified. As such, the subject nucleic acids may be identified by a variety of methods known to one of skill in the art. Similar methods are used to identify host cell cultures in monoclonal antibody production using hybridoma technology (Harlow et al., Antibodies: A Laboratory Manual, First Edition (1988) Cold spring Harbor, N.Y.), and rely on an “addressable” host cell and an “addressable” monoclonal antibody, such that once a monoclonal antibody of interest is identified, a host cell address may be determined and the nucleic acid encoding the antibody of interested isolated from the cell.

For example, the nucleic acids encoding a monoclonal antibody of interest may be recovered, characterized and manipulated from a cell expressing the antibody using techniques familiar to one of skill in the art (Ausubel, et al, Short Protocols in Molecular Biology, 3rd ed., Wiley & Sons, (1995) and Sambrook, et al, Molecular Cloning: A Laboratory Manual, Third Edition, (2001) Cold Spring Harbor, N.Y.).

Methods of Producing a Monoclonal Antibody of Interest

The invention provides several methods of producing a monoclonal antibody of interest. In general these methods involve incubating a host cell containing a nucleic acid encoding a monoclonal antibody of interest under conditions sufficient for production of the antibody. In many embodiments, the methods of producing a monoclonal antibody of interest involve culturing the hybridoma cell to produce the antibody or transferring identified heavy and light chain-encoding nucleic acids for an monoclonal antibody of interest into a suitable vector, and transferring the recombinant vector into a host cell to provide for expression of the monoclonal antibody. In some embodiments, the subject methods involve transferring at least the variable domain-encoding sequences from the identified heavy and light chains into vectors suitable for their expression in immunoglobulin heavy and light chains. Suitable constant domain-encoding sequences and/or other antibody domain-encoding sequences may be added to the variable domain-encoding sequences at this point. These nucleic acid modifications may also allow for humanization of the subject antibody.

The subject monoclonal antibodies can be produced by any method known in the art for the synthesis of antibodies, in particular, by recombinant expression techniques.

Recombinant expression of a subject monoclonal antibody, or fragment, derivative or analog thereof, usually requires construction of an expression vector containing a polynucleotide that encodes the antibody. Methods which are well known to those skilled in the art can be used to construct expression vectors containing antibody coding sequences and appropriate transcriptional and translational control signals. These methods include, for example, in vitro recombinant DNA techniques and synthetic techniques. As such, the invention provides vectors comprising a nucleotide sequence encoding an antibody molecule of the invention.

The expression vector is transferred to a host cell by conventional techniques and the transfected cells are then cultured to produce a subject antibody. In most embodiments, vectors encoding both the heavy and light chains are co-expressed in the host cell to provide for expression of the entire immunoglobulin molecule.

Depending on the constant regions and other regions used, several types of antibody that are known in the art may be made by this method. As well as full length antibodies, antigen-binding fragments of antibodies may be made. These fragments include, but are not limited to, Fab, Fab′ and F(ab′)₂, Fd, single-chain Fvs (scFv), single-chain immunoglobulins (e.g., wherein a heavy chain, or portion thereof, and light chain, or portion thereof, are fused), disulfide-linked Fvs (sdFv), diabodies, triabodies, tetrabodies, scFv minibodies, Fab minibodies, and dimeric scFv and any other fragments comprising a V_{L} and a V_{H} domain in a conformation such that a specific antigen binding region is formed. Antibody fragments, including single-chain antibodies, may comprise the variable region(s) alone or in combination with the entire or partial of the following: a heavy chain constant domain, or portion thereof, e.g., a CH1, CH2, CH3, transmembrane, and/or cytoplasmic domain, on the heavy chain, and a light chain constant domain, e.g., a Ckappa or Clambda domain, or portion thereof on the light chain. Also included in the invention are any combinations of variable region(s) and CH1, CH2, CH3, Cₖₐₚₚₐ, C_{lambda}, transmembrane and cytoplasmic domains.

A variety of host-expression vector systems may be utilized to express a subject monoclonal antibody. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells etc.) harboring recombinant expression constructs containing promoters derived from the genome of mammalian cells (e.g., metallothionein promoter) or from mammalian viruses (e.g., the adenovirus late promoter; the vaccinia virus 7.5K promoter). In many embodiments, bacterial cells such as Escherichia coli, and eukaryotic cells are used for the expression of entire recombinant antibody molecules. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express antibodies. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized to express a subject antibody. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). The efficiency of expression may be enhanced by the inclusion of appropriate transcription enhancer elements, transcription terminators, etc. (see Bittner et al., Methods in Enzymol. 153:51-544 (1987)).

For long-term, high-yield production of recombinant antibodies, stable expression may be used. For example, cell lines, which stably express the antibody molecule may be engineered. Rather than using expression vectors which contain viral origins of replication, host cells can be transformed with immunoglobulin expression cassettes and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into a chromosome and grow to form foci which in turn can be cloned and expanded into cell lines. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt-cells, respectively. Also, antimetabolite resistance can be used as the basis of selection for the following genes: dhfr, which confers resistance to metbotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Phamacol. Toxicol. 32:573-596 (1993); Mulligan, Science 260:926-932 (1993); and Morgan and Anderson, Ann. Rev. Biochem. 62:191-217 (1993); TIB TECH 11(5):155-215 (1993)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981).

The host cell may be co-transfected with two expression vectors of the invention, the first vector encoding a heavy chain derived polypeptide and the second vector encoding a light chain derived polypeptide. The two vectors may contain different selectable markers and origins of replication, which enable equal expression of heavy and light chain polypeptides. Alternatively, a single vector may be used which encodes, and is capable of expressing, both heavy and light chain polypeptides.

Once an antibody molecule of the invention has been produced, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In many embodiments, antibodies are secreted from the cell into culture medium and harvested from the culture medium.

Utility

The invention provides, inter alia, methods for producing a plurality of monoclonal antibodies that each specifically bind to a different antigen. These methods have several uses, many of which will be described below.

In one embodiment, the invention provides methods of treating a subject with a monoclonal antibody of interest. In general these methods involve administering a monoclonal antibody identified by the methods described above to a host in need of treatment. In many embodiments, the monoclonal antibody is a therapeutic monoclonal antibody.

By treatment is meant at least an amelioration of a symptom associated with the pathological condition afflicting the host, where amelioration is used in a broad sense to refer to at least a reduction in the magnitude of a parameter, e.g., symptom, associated with the pathological condition being treated. As such, treatment also includes outcomes where the pathological condition, or at least symptoms associated therewith, are completely inhibited, e.g., prevented from happening, or stopped, e.g., terminated, such that the host no longer suffers from the pathological condition, or at least the symptoms that characterize the pathological condition.

A variety of hosts are treatable according to the subject methods. Generally such hosts are mammals or mammalian, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans. In other embodiment, the host will be an animal model for a human disease.

Of particular interest is treatment and prevention of diseases, conditions and disorders associated with abnormal expression of a cellular protein, usually present on the surface of a cell, e.g., a cancer cell, or conditions associated with secreted proteins.

The methods and compositions of the invention have several research applications. In one exemplary application, the individual antibodies of the plurality of monoclonal antibodies is deposited onto an array or microarray (e.g., using a method provided by U.S. Pat. Nos. 6,372,483, 6,352,842, 6,346,416 and 6,242,266), and labeled samples (e.g., cell extracts or proteins) or pairs of differentially labeled are incubated with the array. Such experiments may provide monoclonal antibodies and antibody-encoding polynucleotide sequences that differentially bind to samples. In one exemplary experiment, cancerous cells or extracts thereof are labeled and incubated with an array of monoclonal antibodies. After washing of the array, data representing the amount of binding of the cell or extract thereof may be extracted for each antibody. Comparison of this data to data generated using normal or non-cancerous cells incubated with a similar or the same array may reveal monoclonal antibodies that specifically recognize the cancer cell. Such antibodies have therapeutic applications.

The methods and compositions of the invention provide specific reagents that can be used in standard diagnostic procedures. For example, the antibodies or their immunoreactive fragments can be employed in immunoassays for detection of target antigens. To perform a diagnostic method, on of the compositions of the invention is provided as a reagent to detect a target antigen in a sample with which it reacts. Procedures for performing immunoassays are well established in the art and hence are not described here.

The human monoclonal antibodies generated by the subject methods may also be used for treatment or prevention of diseases and conditions. The monoclonal antibodies may be used to modulate the activities of target antigens that play a central role in disease development and/or progression. For example, a humanized anti-Her2 antibody, available commercially under the trademark HERCEPTIN®, which selectively inhibits growth of human breast cancer cells, is now employed as a potent drug to treat tens and thousands of breast cancer patients who overexpress the breast cancer antigen Her2.

A further use for the antibodies is in protein expression profiling, where antibodies against a plurality of different antigens may be used to identify specific antigens that are induced in specific condition, or at specific times or places during normal or abnormal development.

Kits

Also provided by the subject invention are kits for practicing the subject methods, as described above. The subject kits at least include one or more of: a plurality of monoclonal antibodies, a cell population producing a plurality of antigens, polyclonal antisera against a plurality of antigens, an animal comprising a cell population expressing a plurality of antigens etc. Other optional components of the kit include: components for performing antibody screening assays, e.g., microtiter plates and ELISA reagents; buffers, nucleotides and reagents for performing hybridomas; and control antibodies for screening assays. The various components of the kit may be present in separate containers or certain compatible components may be precombined into a single container, as desired.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

Also provided by the subject invention is are kits including at least a computer readable medium including programming as discussed above and instructions. The instructions may include installation or setup directions. The instructions may include directions for use of the invention with options or combinations of options as described above. In certain embodiments, the instructions include both types of information.

Providing the software and instructions as a kit may serve a number of purposes. The combination may be packaged and purchased as a means for producing rabbit antibodies that are less immunogenic in a non-rabbit host than a parent antibody, or nucleotide sequences them.

The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc, including the same medium on which the program is presented.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Rabbit Monoclonal Antibodies Raised Against Integrin beta-6

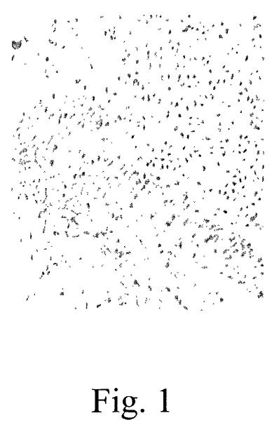

In the following experiment, rabbit monoclonal antibodies against the human integrin beta-6 were obtained. Purified extracellular domain (ECD) of integrin beta-6 was used to immunize rabbits. A total of 4 immunizations were performed, using 200 μg of proteins for each of the immunization in the presence of Freund's adjuvants. Fusions of the rabbit spleen cells and plasmacytoma cell line 240E was performed according to Spieker-Polet (Proc Natl Acad Sci U S A 92(20): 9348-52). Partial screening of hybridoma supernatants in ELISA identified positive clones that secrete antibodies against the specific antigen. Limited dilution and cloning was performed for a subset of these clones. One clone B1 was used to produce monoclonal antibodies that showed positive binding in immunocytochemistry (FIG. 1), Western Blot and FACS analysis and ELISA.

### Example 2

### Expression of Protein X in 240E Cell and Immunization of Rabbits with 240E Cell Expressing Protein X

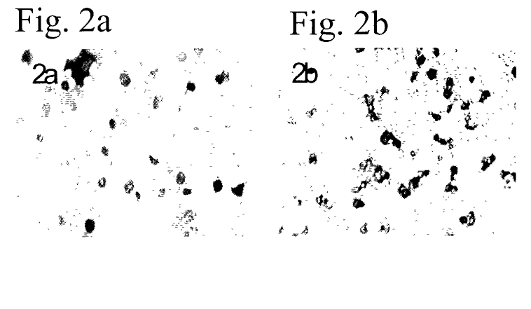

240E cells were transfected with an expression plasmid containing cDNA encoding protein X and immunized the rabbits with transfected cells. Antiserum from the rabbits of transfected cell immunization showed positive titer (FIG. 2) but not 240E mock transfected immunization.

Specific binding was observed with antiserum from the rabbit immunized the 240E cells transfected with Protein X cDNA, but not with pre-immune antiserum or antiserum from the rabbit immunized with control 240E cells (not shown).

### Example 3

### Syngenic Rabbit Cell Line 240E Transfection and Protein Expression

Cell lines. 240E cells do not induce antibody response when used to immunize rabbits. 240E cells may be used as the antigen expressing cell as well as the fusion partner in hybridoma fusion.

Transfection protocol. Different transfection protocols such as lipofection, calcium phosphate, and electroporation are tested to identify the maximal transfection efficiency in 240E cells. Green fluorescent protein (GFP) is used to visualize the transfected cells. To further improve the transfection efficiency, fine-tuning of transfection conditions are performed using a placental secreted alkaline phosphatase (SEAP) expression plasmid as a reporter gene. SEAP assay can quantitatively determine the relative level of protein expression. The level of protein expression, consistency and reproducibility is assessed in this experiment.

Retroviral infection protocol. The ViraPort retroviral expression system (Stratagene) is used to assess the infection efficiency of rabbit cell lines by retroviral vectors. The vector pFB-hrGFP is used. High multiplicity of infection (MOIs) for which 100% of the cells are transfected are desired. In this case, multiple copies of infected retroviral gene are expected to integrate into the genome of the rabbit cells. Repeated infection of the same cells may result in higher copy number of integrants and thus higher level of expression.

### Example 4

### cDNA Clones Expressing Human Proteins

Several different cDNA clones encoding proteins with different sub-cellullar localizations are tested. cDNA clones encoding proteins of research interest once antibodies are generated are used.

A total of 12 proteins expressed in different sub-cellular locations are used. These cDNAs are cloned into a mammalian expression vector. A 6-His tag is fused to the C-termini of the proteins in order to monitor the expression level. The genes that we will use are shown in Table 1.

TABLE 1
A total of 12 genes tested in “multiplex” immunization

| Gene ID | Gene Name | subcellular localization |
|---|---|---|
| M57627 | interleukin 10 [IL10], SUPPRESSOR FACTOR FOR TH1 | secreted |
| U43368 | Vascular endothelial growth factor B | secreted |
| X16323 | Hepatocyte growth factor (hepapoietin A) | secreted |
| X57025 | insulin-like growth factor I | secreted |
| M99487 | Human prostate-specific membrane antigen (PSM) mRNA | transmembrane |
| S66896 | squamous cell carcinoma antigen | transmembrane |
| U06452 | antigen MART-1, melanoma | transmembrane |
| AF036581 | Homo sapiens tumor necrosis factor superfamily member LIGHT mRNA | transmembrane |
| S72904 | APK1 antige, human, ovarian carcinoma cell line OVCAR-3 | cytosolic |
| AF039136 | Homo sapiens Fas binding protein (hDaxx) | cytosolic |
| Z23115 | H. sapiens bcl-xL mRNA | cytosolic |
| K02581 | thymidine kinase, cytosolic | cytosolic |

### Example 5

### Immunization of Multiple Antigens

To generate antibodies for a large number of proteins, it is desirable to use multiple antigens to immunize a single animal to reduce the number of animals used and to save time and effort in most of the procedures. In this scenario, one immunization and one hybridoma fusion will be used to generate monoclonal antibodies for multiple antigens.

The selected 12 cDNA clones are used to transfect 240E cells. The transfected cells are combined into a mixture and injected to rabbits subcutaneously. The transfection efficiency and expression level of the proteins is examined by cell-ELISA on a small portion of the transfected cells using anti-His tag antibodies. Antisera are collected after 3 immunizations. Positive binding of antisera to Cos-7 cells transfected with each of the 12 cDNAs individually reveals the presence of antibodies for each of the 12 proteins.

The antigen expressing cells are immunized to rabbits in standard protocol, variations of which are described below:

A total of 10⁶, 10⁷ and 10⁸ cells is used for each rabbit in duplicate animals in standard whole-cell immunization protocol. Titer of the antisera from different rabbits is compared by cell-ELISA. Briefly, Cos-7 cells transfected with the 12-cDNA as a pool are fixed in situ and stained by serially diluted antisera. Peroxidase-conjugated anti-rabbit antibodies are used to stain the cells bound by primary antibodies. The rabbit with the highest titer indicates a maximal antibody response. Individually transfected cells are also used to estimate the representation of the polyclonal antibodies for individual antigens.

An immunization schedule with 1, 2 and 3-week intervals is carried out. Optimal injection schedule is determined by testing polysera using cell-ELISA.

The survey of different adjuvants (e.g., Freund's vs. special formulated adjuvants) may boost the immune response to antigens displaying on 240E cells. Commercially available adjuvants are tested.

### Example 6

### Hybridoma Generation

Fusions of the rabbit spleen cells and plasmacytoma cell line 240E are performed according to Spieker-Polet, supra. After fusion, the cells are grown in medium containing HAT to select hybridoma. After 2-3 weeks, supernatants from the hybridoma cells are screened for IgG that binds to one of the 12 proteins expressed by the 12-cDNAs. This is accomplished by transfecing Cos-7 cells with the cDNA and subsequently screening the hybridoma supernatants on the transfected Cos-7 cells using cell-ELISA. The number of antigen-specific hybridoma clones predicts the degree of success to generate monoclonal antibodies from transfected cells. The distribution of the monoclonal antibodies to each of the antigens should also indicate the degree of immunodominance.

### Example 7

### Hybridoma Screening

A step in this method of monoclonal antibody production is matching a hybridoma cell and the antibody it produces to an antigen.

After hybridoma colonies emerge, supernatants are collected and screened. Two rounds of screening are performed. In the first round, Cos-7 cells transfected with the pool of 12-cDNA are seeded in 96 well-plates and fixed. Hybridoma supernatants are used to stain the cells. If a hybridoma supernatant contains antibodies specific for one of the 12 antigens, a positive cell ELISA readout will be scored. Such a hybridoma is expanded into 6-well culture plate and more supernatant is collected. In the second round of the screening, hybridoma supernatants selected from the first round are used to stain Cos-7 cells transfected individually with each of the 12 cDNAs. The antigen recognized by each hybridoma antibody may be identified from the positive binding of the monoclonal antibodies to the transfected cells. Limiting dilution may be needed to select a stable clone secreting antibodies at high concentration. The success rate to get monoclonal antibodies for single antigens of the 12 cDNA is calculated. Higher number of antigens (such as 96 or 384) per immunization are also performed. This strategy leads to the generation of a monoclonal antibody bank against human (or other animals, such as mouse) proteins at the proteomic scale.

It is evident from the above results and discussion that the subject invention provides an important new means for generating monoclonal antibodies. Specifically, the subject invention provides a system for generating monoclonal antibodies for a plurality of antigens. As such, the subject methods and systems find use in a variety of different applications, including research, therapeutic and other applications. Accordingly, the present invention represents a significant contribution to the art.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method of making an animal that produces a population of antibodies, which population comprises a plurality of individual antibodies that each cross react with a different antigen, said method comprising:
administering to an animal an effective amount of greater than five different isolated antigens,
so that said animal produces said population of antibodies.

2. The method according to claim 1, wherein said administering step is administering to an animal an effective amount of greater than 20 different isolated antigens.

3. The method according to claim 2, wherein said administering step is administering to an animal an effective amount of greater than 50 different isolated antigens.

4. The method according to claim 1, wherein said administering step is:
administering to an animal an effective amount of a cellular immunogen made up of a cell population producing a plurality of different antigens that are not native to said cells.

5. The method according to claim 4, wherein said antigens are secreted by said cells, present on the surface of said cells, or present inside said cells.

6. The method according to claim 4, wherein said antigens are polypeptides.

7. The method according to claim 4, wherein said cells are derived from the same species as said host.

8. The method according to claim 7, wherein cells identical to cells of said cellular immunogen except that they do not produce said plurality of different antigens are not immunogenic in said animal.

9. The method according to claim 4, wherein said cells are recombinant cells comprising expression cassettes that include nucleic acids encoding said plurality of antigens.

10. The method according to claim 9, wherein said nucleic acids are cDNA nucleic acids.

11. The method according to claim 9, wherein the encoded products of said nucleic acids are unknown.

12. The method according to claim 9, wherein said nucleic acids are of unknown sequence.

13. The method according to claim 9, wherein said expression cassettes provide for expression of said antigens operably linked to a secretion signal.

14. The method according to claim 9, wherein said expression cassettes provide for expression of said antigens operably linked to a cell surface targeting polypeptide.

15. The method according to claim 9, wherein said expression cassettes comprise an inducible promoter operably linked to said antigen-encoding polynucleotide.

16. The method according to claim 4, wherein said plurality of antigens is greater than 10 antigens.

17. The method according to claim 4, wherein said animal is a rabbit, and said cell is a 240E cell.

18. An cell population producing a plurality of antigens that are not native to cells of said cell population.

19. A non-human animal comprising antibodies that specifically bind to a plurality of different isolated antigens.

20. The non-human animal according to claim 19, wherein the non-human animal comprises the cell population producing a plurality of antigens that are not native to cells of said cell population.

21. A composition comprising polyclonal antisera harvested from the non-human animal of claim 19.

22. A method of identifying a plurality of monoclonal antibodies of interest, said method comprising:
(a) obtaining a population of monoclonal antibodies using antibody producing cells of the non-human animal of claim 19; and
(b) screening said population of monoclonal antibodies to identify a plurality of monoclonal antibodies of interest.

23. The method according to claim 22, wherein said plurality of monoclonal antibodies of interest is a plurality of monoclonal antibodies that each specifically bind a different polypeptide antigen.

24. The method according to claim 22, wherein said screening step (b) involves mixing of antigens into pools of antigens.

25. The method according to claim 22, wherein said plurality of monoclonal antibodies is more than about 10 monoclonal antibodies.

26. The method according to claim 22, wherein said obtaining comprises generating hybridoma cells from said antibody producing cells and the harvesting monoclonal antibodies from said produced hybridoma cells.

27. A method of identifying a monoclonal antibody of interest, said method comprising:
(a) obtaining a population of monoclonal antibodies using antibody producing cells of the non-human animal of claim 19; and
(b) screening said population of monoclonal antibodies to identify said monoclonal antibody of interest.

28. A method of identifying a nucleic acid encoding a monoclonal antibody of interest, said method comprising:
(a) identifying a monoclonal antibody of interest according to the method of claim 27; and,
(b) identifying a nucleic acid encoding said monoclonal antibody of interest.
